(19) United States
(12) Patent Application Publication
(10) Pub. No.: US 2011/0151443 A1
(43) Pub. Date: Jun. 23, 2011

(54) MARKER FOR GASTRIC CANCER

(75) Inventors: Joseph Jao Yiu Sung, Ma On shan (CN); Jun Yu, Shatin (CN); Kin Fai Cheung, Shatin (CN)

(73) Assignee: The Chinese University of Hong Kong Room 328, Pi Ch'iu Building, Shatin (HK)

(21) Appl. No.: 12/646,839

(22) Filed: Dec. 23, 2009

(51) Int. Cl. C12Q 1/68; C07H 21/04

(52) U.S. Cl. 435/6; 536/23.1

(57) ABSTRACT

Methods for detecting and assessing the progress and prognosis of gastric cancer are presented. Primers and probes for use in the methods are also disclosed as are methods for suppressing gastric cancer and kits for implementing the methods.

### FIELD

The subject matter disclosed generally relates to markers for gastric cancer, and methods for the inhibition of gastric cancer.

### BACKGROUND

The following publications are noted:

US 2007/0259368 A1 published Nov. 8, 2007 to Sungwhan An et al. Discloses an epigenetic marker for gastric cancer and a method for discovering a methylation marker gene for the conversion of a normal cell to gastric cancer cell.

US 2005/0026183 A1 published Feb. 3, 2005 to Jian-Bing Fan et al. Discloses a method for identification of differentially methylated genomic CpG dinucleotide sequences associated with cancer.

Kin-Fai Cheung, et al., “Epigenetic Characterization of a Novel Tumor Suppressor Gene, RNF180,” in Gastric Cancer and Its Application in Noninvasive Cancer Detection” Gastroenterology 134, Supplement 1, p382, April 2008. Describes an association between RNF180 gene expression and gastric cancer.

Cheung et al, “Epigenetic Characterization of a Novel Tumor Suppressor Gene, RNF 180, in Gastric Cancer and its Application in Noninvasive Cancer Detection” (poster) presented at Digestive Disease Week in San Diego, May 18-21, 2008. Discloses portions of the sequence of the RNF180 gene and aspects of its expression pattern and relationship to Gastric Cancer.

### SUMMARY

In a first embodiment, there is disclosed a method for detecting gastric cancer in a biological sample. The method may comprise the step of: detecting methylation of a patient sample sequence of at least 15 consecutive base pairs, within a contiguous sequence at least 95% similar to the region consisting of SEQ ID NO:1; wherein significant methylation level is indicative of cancer presence in the sample.

In alternative embodiments, the target sequence may be at least 50 base pairs long and contains a plurality of CpG base pairs. The method may further comprise comparing the methylation level of the patient sample DNA with methylation level of non-cancerous cells. The determining may comprise treating the sample with a reagent that differentially modifies methylated and unmethylated DNA. The reagent may comprise a restriction enzyme that preferentially cleaves unmethylated DNA. The determining may comprise treating the sample with sodium bisulphate. The determination may be performed by combined bisulfite restriction analysis (COBRA). The sample may be a blood sample. The determining may comprise the steps of: amplifying DNA treated with a restriction enzyme using primers selective for a CpG-containing genomic sequence, the genomic sequence may be contained within SEQ ID NO:1; and comparing the level of the amplified portion of the genomic sequence in unknown samples to compare the methylation level with non-cancerous sample to thereby detect the presence of gastric cancer. The reagent may comprise an enzyme that preferentially cleaves unmethylated DNA. The amplifying may use the polymerase chain reaction. The detecting may use a primer or probe selected from the group consisting of: SEQ ID NOS:3, 4, 5, 6, 7, 8, 9, 10, 11, 12, 13, 14, 15, 16, 17, 18, 19, 20, 21, 22, 23, 24, 25, 26, 27, 35, 36, 43, 44 and 46.

In another embodiment, there is disclosed a method for detecting gastric cancer in a biological sample. The method may comprise the step of: a) detecting the level of a target RNA with at least 95% sequence similarity to a region of at least 15 contiguous bases of sequence SEQ ID NO:2 contained in the sample, b) wherein a significantly lower amount of the sequence in the sample relative to a non-cancerous control sample is indicative of presence of gastric cancer in the biological sample.

In alternative embodiments; the region may at least 25 base pairs long. The sample may comprise gastric tissue. The detecting may comprise amplifying the region. The amplifying may use the polymerase chain reaction. The detecting may comprise using a primer or probe selected from the group consisting of: SEQ ID NOS:28, 29, 31, 32, 33, 34, 37 and 38.

In another embodiment, there is disclosed an isolated nucleic acid sequence at least about 10 base pairs long and 95% identical to a fragment of the region between about −202 bp to +372 bp relative to the transcription start site of the RNF180 gene.

In alternative embodiments, there is disclosed an isolated nucleic acid sequence at least about 10 base pairs long and 95% identical to a fragment of SEQ ID NO:1 or SEQ ID NO:2.

In alternative embodiments, the isolated sequence may be at least about 20 base pairs long and at least 99% similar to the corresponding fragment of the region.

In another embodiment, there is disclosed a composition for suppressing gastric cancer. The composition may comprise a biologically acceptable expression vector for expressing a portion of the RNF180 gene in patient's cells.

In alternative embodiments, the vector may be suitable to direct expression of RNF180 protein in patient's cells.

In another embodiment, there is disclosed a method for inhibiting gastric cancer in a subject. The method may comprise exposing the cells of the subject to the composition disclosed herein.

In alternative embodiments, the vector may be delivered by viral transduction.

In another embodiment, there is disclosed a method for assessing the progress of gastric cancer in a subject. The method may comprise the steps of: detecting the level of a target RNA with at least 95% sequence similarity to a region of at least 10 contiguous bases of sequence SEQ ID NO:2 contained in the sample; comparing the results to a reference; and using the results to determine the progress of the gastric cancer in the subject.

In another embodiment, there is disclosed a method for assessing the progress of gastric cancer in a subject. The method may comprise the steps of: detecting methylation of a patient sample sequence of at least 10 consecutive base pairs, within a contiguous sequence at least 95% similar to the region consisting SEQ. ID NO:1; comparing the results to a reference; and using the results to determine the progress of the gastric cancer in the subject.

In another embodiment, there is disclosed a kit for detecting the presence of gastric cancer cells in a biological sample. The kit may comprise: reagents for detecting a significant level of DNA methylation in a nucleic acid sequence having at least 95% sequence identity over 10 contiguous base of the fragment consisting of SEQ ID NO:1.

In another embodiment, there is disclosed a kit for detecting the presence of gastric cancer cells in a biological sample. The kit may comprise: a pair of amplification primers suitable for use to detect RNA transcripts in the sample with at least 95% sequence homology to a contiguous sequence of at least 10 bases of SEQ ID NO:2.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

FIG. 1 is an embodiment of a nucleic acid sequence of a region surrounding the core promoter region of the RNF180 gene, and is designated SEQ ID NO:1. This corresponds to Genbank Accession Number NM_{—}001113561 chr5: 63497153-63497758.

FIG. 2 is an embodiment of a nucleic acid probe sequence for transcripts of the RNF180 gene, suitable to detect transcript variants 1 and 2 of the RNF180 gene under high stringency conditions. It is designated SEQ ID NO:2.

FIG. 3 is an embodiment of a probe for a core promoter region of the RNF180 gene in the embodiment according to FIG. 1. It is designated SEQ ID NO:3.

FIG. 4 is a series of alternative embodiments for probes for the core promoter region of the RNF180 gene (SEQ ID NOS:4-27) according to FIG. 1. Numbering in brackets refers to positions relative to SEQ ID NO:1.

FIG. 5 is an embodiment of a primer pair (SEQ ID NOS:28 and 29) for amplifying RNA sequences of an embodiment.

FIG. 6 shows a series of embodiments for probes for RNA sequences according to FIG. 2 (SEQ ID NO:2).

FIG. 7 is a bisulfite sequencing primer pair (SEQ ID NOS:35 and 36) suitable for amplifying a portion of a promoter sequence of an embodiment.

FIG. 8 is a primer pair (SEQ ID NOS:37 and 38) suitable for amplifying a portion of the mRNA sequence in an embodiment.

FIG. 9 is an immunogen region used for detection of RNF180 protein expression in an embodiment.

FIG. 10 is the 592 amino acid predicted protein sequence (SEQ ID NO:40) encoded by a first transcription variant of the RNF180 gene of an embodiment.

FIG. 11 is the 416 amino acid predicted protein sequence (SEQ ID NO:41) encoded by a second transcription variant of the RNF180 gene of an embodiment.

FIG. 12 is a subsequence of the core promoter region (SEQ ID NO:42) presented in the embodiment of FIG. 1.

FIG. 13 is a primer pair (SEQ ID NOS:43 and 44) for methylation detection suitable to amplify promoter sequences of an embodiment.

FIG. 14 is a portion of the core promoter region (SEQ ID NO:45) according to FIG. 1, showing binding regions for primers and probes according to FIG. 13 and FIG. 3. Primer and probe binding sequences are underlined.

FIG. 15 is a Taqman™ probe sequence (SEQ ID NO:46) corresponding to SEQ ID NO:3 in FIG. 3.

FIG. 16 is a representation of the structure of RNF180 transcript variants, transcription start site (TSS) and functional promoter mapping (SEQ ID NO:47).

FIG. 17 shows mRNA expression and promoter methylation of RNF180 in gastric cancer cell lines.

FIG. 18 shows functional analysis and gene expression of RNF180 in gastric cancer.

FIG. 19 shows promoter methylation of RNF180 analysis in primary tumors and plasma of patients with gastric cancer.

FIG. 20 shows Kaplan-Meier estimates of patient survival with gastric cancers, according to RNF180 protein expression.

### DETAILED DESCRIPTION OF EMBODIMENTS

### Terms

In this disclosure the following terms have the meanings set forth below:

In this disclosure the term “or” is generally employed in its sense including “and/or” unless the content clearly dictates otherwise.

In this disclosure, the term “biomarker” means a substance such as a gene, a measurement of a variable related to a disease that may serve as an indicator or predictor of that disease. Biomarkers are parameters from which the presence or risk of a disease can be inferred, rather than being a measure of the disease itself.

In this disclosure the terms “nucleic acid”, “nucleic acid sequence,” and the like mean polynucleotides, which may be gDNA, cDNA or RNA and which may be single-stranded or double-stranded. The term also includes peptide nucleic acid (PNA), or to any chemically DNA-like or RNA-like material. “cDNA” refers to copy DNA made from mRNA that is naturally occurring in a cell. “gDNA” refers to genomic DNA. Combinations of the same are also possible (i.e., a recombinant nucleic acid that is part gDNA and part cDNA).

In this disclosure the terms “operably associated” and “operably linked,” mean functionally coupled nucleic acid sequences.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are well known in the art and are provided, e.g., in Innis et al. (1990) PCR Protocols, A Guide to Methods and Applications, Academic Press, Inc. N.Y.).

In this disclosure the term “polypeptide” means a polypeptide encoded by a nucleic acid molecule.

In this disclosure the terms “gene expression” and “protein expression” mean and includes any information pertaining to the amount of gene transcript or protein present in a sample, as well as information about the rate at which genes, RNA or proteins are being expressed or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

In this disclosure the term “polypeptide” means a molecule comprised of two or more amino acids, preferably more than three. Its exact size will depend upon many factors.

In this disclosure the term “oligonucleotide” means a molecule comprised of two or more nucleotides, preferably more than three. Its exact size will depend upon many factors which, in turn, depend upon the ultimate function and use of the oligonucleotide. In particular embodiments an oligonucleotide may have a length of about 10 nucleotides to 100 nucleotides or any integer therebetween. In embodiments oligonucleotides may be about 10 to 30 nucleotides long, or may be between about 20 and 25 nucleotides long. In embodiments an oligonucleotide may be greater than about 10, 11, 12, 13, 14, 15, 16, 17, 18, 19, 20, 21, 22, 23, 24, or 25 nucleotides long for specificity. In certain embodiments oligonucleotides shorter than these lengths may be suitable.

In this disclosure the term “primer” means an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA or RNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains at least or more than about 10, or 15, or 20, or 25 or more nucleotides, although it may contain fewer nucleotides or more nucleotides. The factors involved in determining the appropriate length of primer are readily known to one of ordinary skill in the art.

In this disclosure the term “primer pair”, means a pair of primers which hybridize to opposite strands a target DNA molecule, to regions of the target DNA which flank a nucleotide sequence to be amplified.

In this disclosure the term “primer site”, means the area of the target DNA to which a primer hybridizes.

In this disclosure, the nucleic acids, polynucleotides, proteins, and polypeptides described and claimed refer to all forms of nucleic acid and amino acid sequences, including but not limited to genomic nucleic acids, pre-mRNA, mRNA, polypeptides, polypeptides, polymorphic variants, alleles, mutants, and interspecies homologs that:

(1) have or encode an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a referenced nucleic acid or an amino acid sequence described herein;
(2) specifically bind to or encode polypeptides that specifically bind to antibodies, e.g., polyclonal antibodies, raised against an immunogen comprising a referenced amino acid sequence, immunogenic fragments thereof, and conservatively modified variants thereof;
(3) specifically hybridize under stringent hybridization conditions to a disclosed nucleic acid sequence or to a nucleic acid sequence encoding a disclosed amino acid sequence, and conservatively modified variants thereof,
(4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence.

A polynucleotide or polypeptide sequence is typically from a mammal including, but not limited to, primate, e.g., human; rodent, e.g., rat, mouse, hamster; cow, pig, horse, sheep, or any mammal. In particular embodiments the polynucleotide and polypeptide sequences disclosed are from humans. The nucleic acids and proteins of the invention include both naturally occurring or recombinant molecules.

In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., serum, plasma, platelets, red blood cells, and the like), sputum or saliva, lymph and tongue tissue, cultured cells, e.g., primary cultures, explants, and transformed cells, stool, urine, stomach biopsy tissue etc. A biological sample is typically obtained from a eukaryotic organism, may be a mammal, may be a primate and may be a human subject.

In this disclosure the term “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., tongue, colon, prostate, kidney, bladder, lymph node, liver, bone marrow, blood cell, stomach tissue, etc.) among other factors. Representative biopsy techniques include, but are not limited to, excisional biopsy, incisional biopsy, needle biopsy, surgical biopsy, and bone marrow biopsy. A wide range of biopsy techniques are well known to those skilled in the art who will choose between them and implement them with minimal experimentation.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamide) containing restriction-digested genomic DNA is not to be considered an isolated nucleic acid.

In this disclosure a “cell” may be isolated, may be comprised in a group of cells, may be in culture, or may be comprised in a living subject and may be a mammalian cell and may be a human cell. Similarly “tissue” may comprise any number of cells and may be comprised in a living subject more may be isolated therefrom.

In this disclosure “cancer” means and includes any malignancy, or malignant cell division or malignant tumour, or any condition comprising uncontrolled or inappropriate cell proliferation and includes without limitation any disease characterized by uncontrolled or inappropriate cell proliferation.

In this disclosure the terms “gastric cancer” and “stomach cancer” have the same meaning and mean a cancer of the stomach or of stomach cells. Such cancers may be adenocarcinomas that occur in the lining of the stomach (mucosa) and may be in pylorus, body or cardial (lower, body and upper) parts of the stomach.

In this disclosure the term “gastric cancer cell” means a cell characteristic of gastric cancer, and includes cells which are precancerous.

In this disclosure the term “precancerous” means a cell which is in the early stages of conversion to a cancer cell or which is predisposed for conversion to a cancer cell. Such cells may show one or more phenotypic traits characteristic of the cancerous cell.

In this disclosure the term “purified,” means nucleic acids or polypeptides separated from their natural environment so that they are at least 95% of total nucleic acid or polypeptide in a given sample. Protein purity is assessed herein by SDS-PAGE and silver staining. Nucleic acid purity is assessed by agarose gel and EtBr staining.

In this disclosure the terms “purified” and “substantially purified,” mean nucleic acid or protein sequences that are removed from their natural environment and may be at least 75% pure. Preferably, at least about 80, 85, 90 or 95% purity is attained.

In this disclosure the term “detection” means any process of observing a marker, or a change in a marker (such as for example the change in the methylation state of the marker, or the level of expression of nucleic acid or protein sequences), in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation and may be based on a comparison with one or more control samples. It will be understood that detecting a gastric cancer as disclosed herein includes detecting precancerous cells that are beginning to or will, or have an increased predisposition to develop into gastric cancer cells. Detecting a gastric cancer also includes detecting a likely probability of mortality or a likely prognosis for the condition.

In this disclosure the term “expression vector” means a replicable DNA construct used to express DNA which encodes a desired protein or RNA sequence and which includes a transcriptional unit comprising an assembly of (1) genetic element(s) having a regulatory role in gene expression, for example, promoters, operators, or enhancers, operatively linked to (2) a DNA sequence encoding a desired protein (in this case, an RNF180 protein) which is transcribed into mRNA and translated into protein, and (3) appropriate transcription and translation initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome or in the form of viral sequences which may or may not integrate into the chromosomes. A wide range of expression vectors will be readily recognised and used by those skilled in the art.

In this disclosure the terms “homology”, “identity” and “similarity” mean sequence similarity between two peptides or between two nucleic acid molecules. They can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated or “non-homologous” shares less than 40% identity, preferably less than 25% identity with a sequence of the present invention. In comparing two sequences, the absence of residues (amino acids or nucleic acids) or presence of extra residues also decreases the identity and homology/similarity. In particular embodiments two or more sequences or subsequences may be considered substantially or significantly homologous, similar or identical if their sequences are about 60% identical, or are about 65%, 70%, 75%, 80%, 85%, 90%, 91%, 92%, 93%, 94%, 95%, 96%, 97%, 98%, 99%, or higher over a specified region, when compared and aligned for maximum correspondence over a comparison window or designated region, as measured using a BLAST or BLAST 2.0 sequence comparison algorithms with default parameters described below, or by manual alignment and visual inspection such as provided on-line by the National Center for Biotechnology Information (NCBI). This definition also refers to, or may be applied to, the compliment of a test sequence. The definition also includes sequences that have deletions and/or additions, as well as those that have substitutions. In embodiments identity exists over a region that is at least about 10, 11, 12, 13, 14, 15, 16, 17, 18, 19, 10, 21, 22, 23, 24, 25 or more amino acids or nucleotides in length, or over a region that is more than about 10, 15, 20, 25, 30, 35, 40, 45, 50, 55, 60, 65, 70, 75, 80, 85, 90, 95 or more than about 100 amino acids or nucleotides in length.

In this disclosure the term “methylation-sensitive PCR” (i.e., MSP) means a polymerase chain reaction in which amplification of the compound-converted template sequence is performed. Two sets of primers are designed for use in MSP. Each set of primers comprises a forward primer and a reverse primer. One set of primers, called methylation-specific primers (see below), will amplify the compound-converted template sequence if C bases in CpG dinucleotides within the vimentin DNA are methylated. Another set of primers, called unmethylation-specific primers (see below), will amplify the compound-converted template sequences if C bases in CpG dinucleotides within the vimentin DNA are not methylated.

In this disclosure the terms “inhibit” and “suppress” where used with reference to cancer cells or the growth or development thereof, mean and include any effects that result in or comprise slowing or preventing growth or cell division of the cells, killing the cells, disabling the cells, and in any way reducing the viability, rate of division or longevity of the cells and includes any metabolic changes which change the characteristics of the cells in ways more characteristic of benign rather than malignant cell populations.

In this disclosure “Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized. Antibodies exist, e.g., as intact immunoglobulins or as a number of well-characterized fragments which can be produced by digestion with various peptidases. The term antibody, as used herein, includes both complete antibodies and also antibody fragments either produced by the modification of whole antibodies, or synthesized de novo using recombinant DNA methodologies (e.g., single chain Fv) or those identified using phage display libraries.

In this disclosure the term “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein.

In this disclosure the term “amplify”, means a process whereby multiple copies are made of one particular locus of a nucleic acid, such as genomic DNA or cDNA. Amplification can be accomplished using any one of a number of known means, including but not limited to the polymerase chain reaction (PCR), transcription based amplification and strand displacement amplification (SDA).

In this disclosure the term “polymerase chain reaction” or “PCR”, means, a technique in which cycles of denaturation, annealing with primer, and extension with DNA polymerase are used to amplify the number of copies of a target DNA sequence by approximately 10⁶ times or more. The polymerase chain reaction process for amplifying nucleic acid is covered by U.S. Pat. Nos. 4,683,195 and 4,683,202.

In this disclosure the term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

In this disclosure a “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

In this disclosure the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

DNA methylation involves the addition of a methyl group to the 5 position of cytosine (C), typically (but not necessarily) in the context of CpG (a cytosine followed by a guanine) dinucleotides. As used herein, “an increased methylation level” or “a significant methylation level” refers to the presence of at least one methylated C nucleotide in a DNA sequence where the corresponding C is not methylated in a normal control sample (such as a DNA sample extracted from a non-cancerous cell or tissue sample), in some cases at least 2, 3, 4, 5, or more Cs are methylated at locations where the Cs are unmethylated in the control DNA.

### Exclusion of Certain Sequences:

It will be understood that in particular embodiments individual examples of sequences, probes, primers, polypeptides or the like may be excluded.

### Detection of Nucleic Acids and Polypeptides:

A range of methods for the detection of specific nucleic acid sequences and polypeptides and their application will be readily apparent to those skilled in the art.

Nucleic acid molecules and polypeptides can be detected using a number of different methods. Methods for detecting nucleic acids include, for example, PCR and nucleic acid hybridizations (e.g., Southern blot, Northern blot, or in situ hybridizations). Specifically, oligonucleotides (e.g., oligonucleotide primers) capable of amplifying a target nucleic acid can be used in a PCR reaction. PCR methods generally include the steps of obtaining a sample, isolating nucleic acid (e.g., DNA, RNA, or both) from the sample, and contacting the nucleic acid with one or more oligonucleotide primers that hybridize(s) with specificity to the template nucleic acid under conditions such that amplification of the template nucleic acid occurs. In the presence of a template nucleic acid, an amplification product is produced. Conditions for amplification of a nucleic acid and detection of an amplification product are known to those of skill in the art. A range of modifications to the basic technique of PCR also have been developed, including but not limited to anchor PCR, RACE PCR, RT-PCR, and ligation chain reaction (LCR). A pair of primers in an amplification reaction must anneal to opposite strands of the template nucleic acid, and should be an appropriate distance from one another such that the polymerase can effectively polymerize across the region and such that the amplification product can be readily detected using, for example, electrophoresis. Oligonucleotide primers can be designed using, for example, a computer program such as OLIGO (Molecular Biology Insights Inc., Cascade, Colo.) to assist in designing primers that have similar melting temperatures. Typically, oligonucleotide primers are 10 to 30 or 40 or 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), but can be longer or shorter if appropriate amplification conditions are used.

In this disclosure the term “standard amplification conditions” refers to the basic components of an amplification reaction mix, and cycling conditions that include multiple cycles of denaturing the template nucleic acid, annealing the oligonucleotide primers to the template nucleic acid, and extension of the primers by the polymerase to produce an amplification product.

Detection of an amplification product or a hybridization complex is usually accomplished using detectable labels. The term “label” with regard to a nucleic acid is intended to encompass direct labeling of a nucleic acid by coupling (i.e., physically linking) a detectable substance to the nucleic acid, as well as indirect labeling of the nucleic acid by reactivity with another reagent that is directly labeled with a detectable substance. Detectable substances include various enzymes, prosthetic groups, fluorescent materials, luminescent materials, bioluminescent materials, and radioactive materials. Examples of suitable enzymes include horseradish peroxidase, alkaline phosphatase, beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. An example of indirect labeling includes end-labeling a nucleic acid with biotin such that it can be detected with fluorescently labeled streptavidin.

Specific polypeptide sequences may be detected using polyclonal or monoclonal antibodies which can be prepared in conventional ways as will be readily understood and applied by those skilled in the art. Those skilled in the art will readily identify and prepare and raise antibodies to desirable polypeptide sequences to implement the subject matter disclosed and claimed.

The term “probe” with regard to nucleic acid sequences is used in its ordinary sense to mean a selected nucleic acid sequence that will hybridise under specified conditions to a target sequence and may be used to detect the presence of such target sequence. It will be understood by those skilled in the art that in some instances probes may be also be useable as primers, and primers may useable as probes.

### Methylation:

In embodiments, DNA methylation alterations can be detected using a number of different methods. Methods for detecting DNA methylation include, for example, methylation-sensitive restriction endonucleases (MSREs) assay by either southern or polymerase chain reaction (PCR) analysis, methylation specific-PCR (MS-PCR), methylation-sensitive single nucleotide primer extension (Ms-SnuPE), bisulifte sequencing, pyrosequencing, methylation-specific single-strand conformation analysis (MS-SSCA), combined bisulifte restriction analysis (COBRA), methylation-specific denaturing gradient gel electrophoresis (MS-DGGE), methylation-specific melting curve analysis (MS-MCA), methylation-specific denaturing high-performance liquid chromatography (MS-DHPLC), methylation-specific microarray (MSO). These assays can be either PCR analysis, quantitative analysis with fluorescence labelling or southern blot analysis.

### Articles of Manufacture

This disclosure encompasses articles of manufacture (e.g., kits) that contain one or more nucleic acid molecules, or one or more vectors that encode a nucleic acid molecule. Such nucleic acid molecules are formulated for administration as described herein, and can be packaged appropriately for the intended route of administration. For example, a nucleic acid molecule or a vector encoding a nucleic acid molecule can be contained within or accompanied by a pharmaceutically acceptable carrier.

Kits of according to embodiments can include additional reagents (e.g., buffers, co-factors, or enzymes). Pharmaceutical compositions according to embodiments can include instructions for administering the composition to an individual. Kits may also contain a control sample or a series of control samples that can be assayed and compared to the biological sample. Each component of a kit may be enclosed within an individual container and all of the various containers are within a single package.

### Compositions and Delivery of Compositions to Target Cells

In certain embodiments there are disclosed compositions for delivery to target cells. It will be understood that the compositions used in particular embodiments may be used in combination with suitable pharmaceutically acceptable carriers or excipients and may be used in any suitable dosage forms. Those skilled in the art will readily identify, select from, and use the foregoing to suit the circumstances in question. Where a cell to be treated is comprised in the body of a subject the methods disclosed may be implemented and the compositions disclosed may be delivered to the cell in any conventional ways including without limitation the delivery of the tetrose or prodrug, orally, parentally, enterally, intramuscularly, subcutaneously, intravenously, or by inhalation and may be delivered in combination with suitable carriers or excipients, in suitable dosage forms including without limitation tablets, capsules, subdermal pumps or other routes useful to achieve an effect. Alternative delivery methods may include osmotic pumps, implantable infusion systems, intravenous drug delivery systems, and refillable implantable drug delivery systems. Delivery by inhalation may comprise delivery using nebulizers, metered dose inhalers, powder inhalers, all of which are familiar to those skilled in the art. Suitable methods, compositions and routes of delivery will be readily recognised and implemented by those skilled in the art.

### EMBODIMENTS

In embodiments there is disclosed the identification of a novel tumor suppressor gene, RNF180, in gastric cancer and use of this novel marker for non-invasive diagnosis and prognostic prediction of gastric cancer. In embodiments it is disclosed that transcriptional silence of RNF180 is associated with its promoter methylation in gastric cancer. In further embodiments it is disclosed that detecting methylated RNF180 DNA in the plasma of gastric cancer patients can be used as a biomarker for detection of gastric cancer and that detecting RNF180 protein expression in gastric tissues is associated with patient survival and can be used as a potential prognositc biomarker. This application also provides primers and methods for assessing the expression of RNF180, the prognosis and progress of gastric cancer, and kits for diagnosing the disease.

### First Embodiment

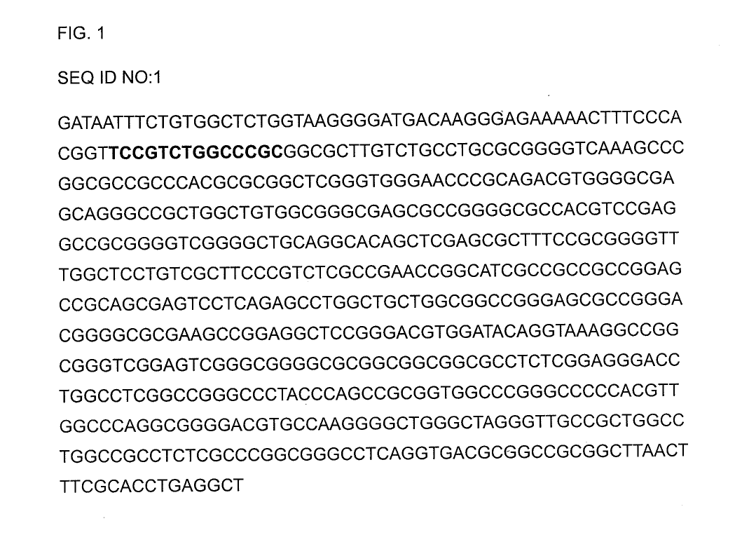

In a first embodiment there are disclosed isolated nucleic acid sequences that hybridise under high stringency with a promoter region of the RNF180 gene consisting of SEQ ID NO:1 (as shown in FIG. 1), representing Genbank accession number: NW_{—}001838935.2 (from nucleotide 5384353 to 5383780). In particular embodiments the sequences may be suitable for use as probes for amplification primers and may be of any suitable length and may be 10 or more nucleotides long, or may be more than about 15, 20, 25, 30, 35, 40, 45 or more than 50 nucleotides long or longer. In embodiments the nucleic acid sequences may have greater than about 95%, 96%, 97%, 98%, or 99% identity to, or may be identical to the corresponding portion of SEQ ID NO:1 or its complementary sequence.

One example of such a probe sequence is presented in FIG. 3 as SEQ ID NO:3 and its corresponding location is indicated by underlining in SEQ ID NO:1 in FIG. 1. In alternative embodiments of this and the other disclosed embodiments probes, such as SEQ ID NO:3 may be constructed as Taqman™ probes. Such an embodiment of SEQ ID NO:3 is presented in FIG. 15 as SEQ ID NO:46.

In variants of the embodiment there is disclosed an isolated nucleic acid sequence at least 15 base pairs long and 95% identical to a fragment of the region between about −202 bp to +372 bp relative to the transcription start site of the RNF180 gene. In alternative embodiments the isolated sequence may be at least 25 base pairs long and at least 95% identical to the corresponding fragment of said region.

FIG. 4 shows a series of alternative probe sequences suitable to specifically bind to SEQ ID NO:1 under high stringency conditions. These are designated SEQ ID NOS:4 through 27.

In an embodiment using SEQ ID NO:3 as a probe for gastric cancer, it may be possible to achieve 91% specificity and 63% sensitivity in discriminating gastric cancer patients from control subjects.

### Second Embodiment

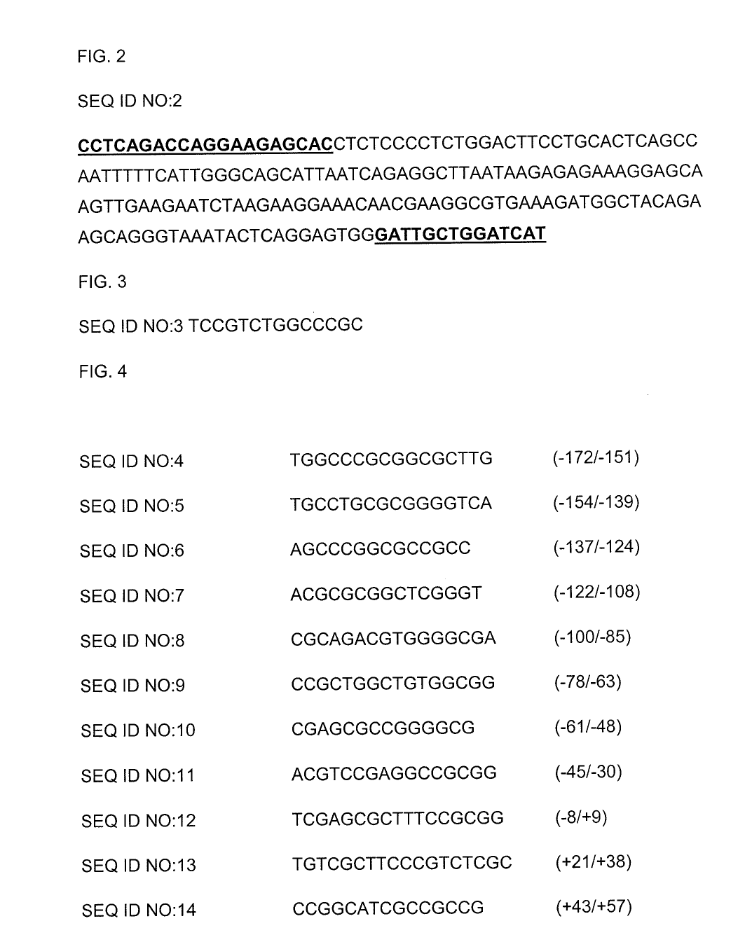

In a second embodiment is disclosed an isolated nucleic acid sequence presented in FIG. 2 as SEQ ID NO:2. SEQ ID NO:2 is the coding sequence common to two mRNA transcripts of RNF180, and represent Genbank Accession numbers NM_{—}001113561 and NM_{—}178532.3 (from nucleotide 1144 to 1334) and referred to as transcription variants 1 and 2. There are further disclosed nucleic acid probes comprising portions of the complement of SEQ ID NO:2 and with sequences suitable to bind to RNF180 mRNA under high stringency conditions.

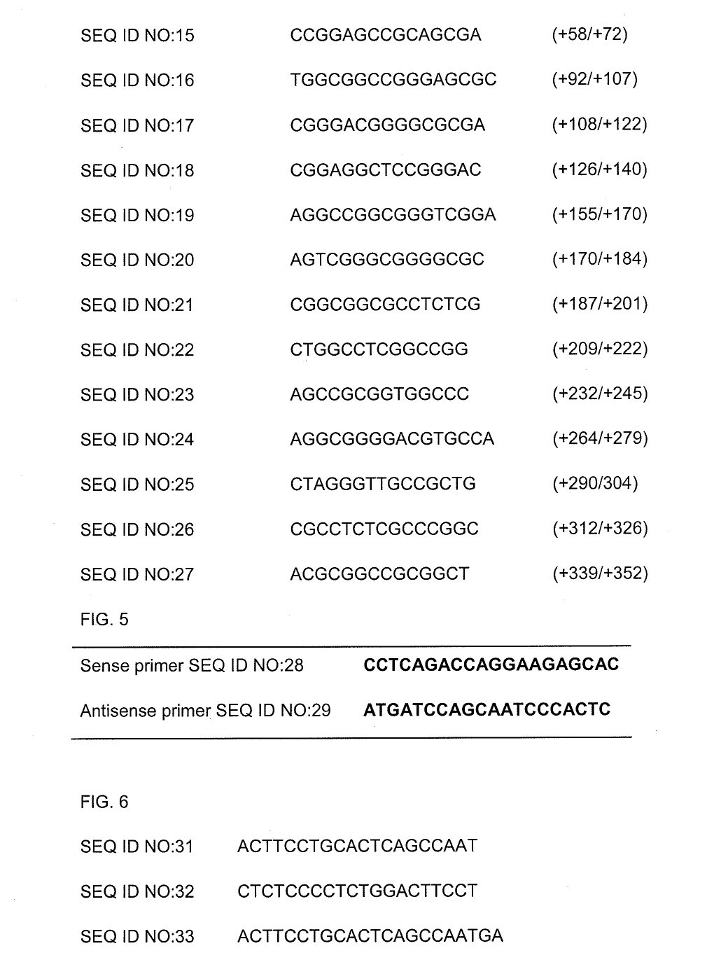

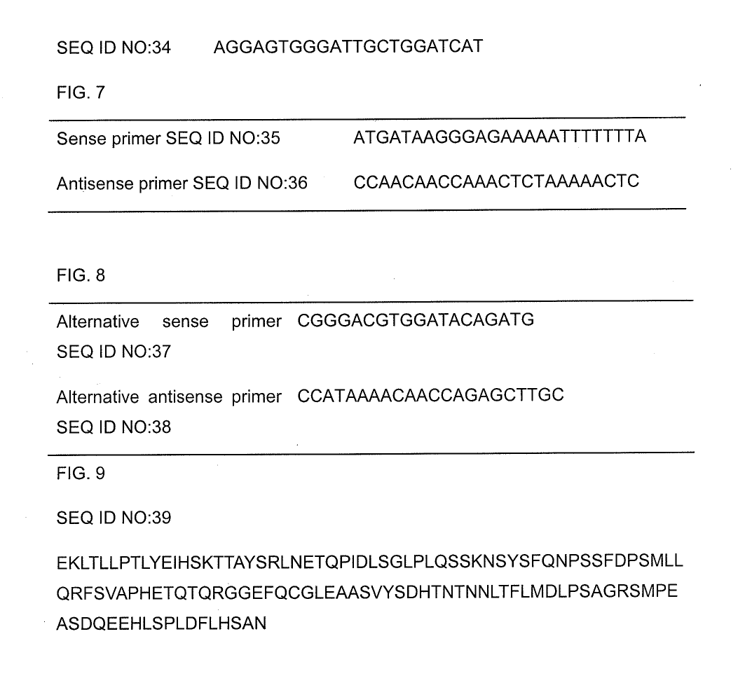

A pair of primers suitable to amplify a portion of SEQ ID NO:2 under high stringency conditions is shown in FIG. 5 and a further alternative primer pair is shown in FIG. 8. It will be understood that the antisense primers may also be useable separately as probes for SEQ ID NO:2 mRNA. FIG. 7 shows the sequence of a number of primers suitable to amplify portions of the mRNA according to SEQ ID NO:2 under high stringency conditions.

The probes of embodiments may be of any suitable length and may be 10 or more nucleotides long, and may be more than about 15, 20, 25, 30, 35 or more then about 40 nucleotides long or longer. In embodiments the nucleic acid sequences may have greater than about 95%, 96%, 97%, 98%, or 99% similarity to, or may be identical to a portion of SEQ ID NO:2.

### Third Embodiment

In a third embodiment there is disclosed an isolated polypeptide encoded by the amino acid sequence represented by SEQ ID NOS:39, 40 and 41. There are further disclosed antibodies specific for the polypeptide or a portion thereof and methods of using the antibodies to detect the presence of gastric cancer cells in a biological sample. Such antibodies may be polyclonal or monoclonal.

In one variant of this embodiment the antibody is a polyclonal anti-human RNF180 antibody. In alternative embodiments suitable monoclonal antibodies may be useable. Such antibodies may include an antibody produced by Abcam™ Limited Cat #ab76803 and by Abnove™ Corporation/Novus™ Biologicals; Cat #H00285671-A01, H00285671-M05

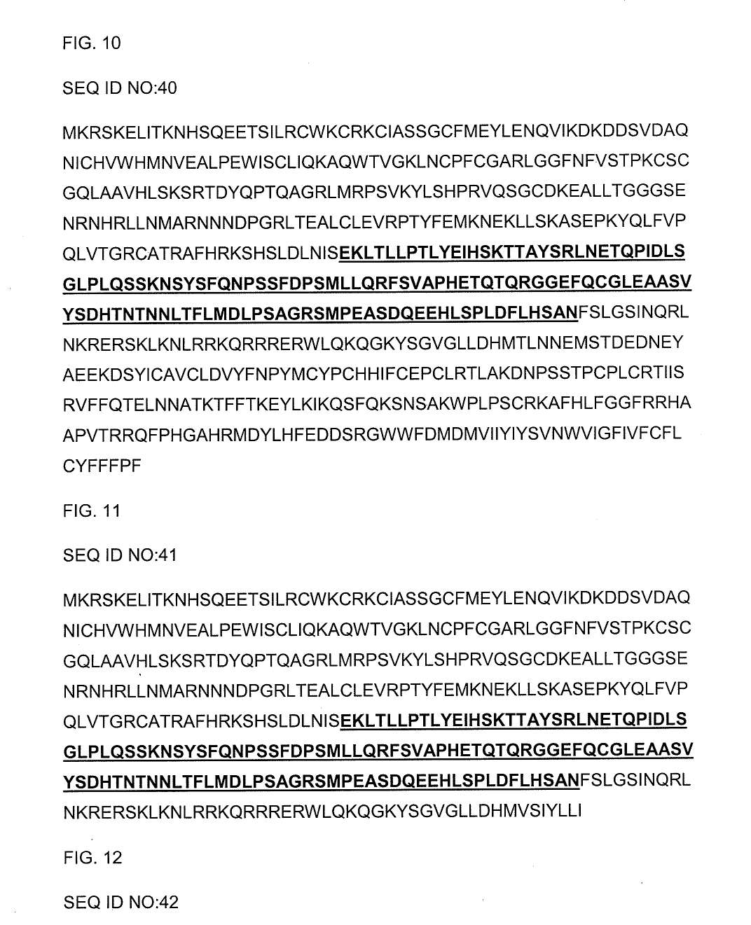

The mRNA sequence presented in FIG. 6 encodes a predicted protein sequence shown in FIG. 9 and in embodiments. Antibodies to this sequence may be used for the purposes of assessing RNF180 protein expression. The full predicted protein translation products of two transcription variants of the RNF180 gene are presented in FIG. 10 and FIG. 11 and in alternative embodiments antibodies to all or selected portions of one or both of these variant sequences may be selected. FIG. 10 shows the sequence of isoform 1 NP_{—}001107033 length=592 amino acid and FIG. 11 shows the sequence of isoform 2 NP_{—}848627 length=416 amino acid. The location of the diagnostic sequence presented in FIG. 9 is underlined in FIG. 10 and FIG. 11.

### Fourth Embodiment

In a fourth embodiment there are disclosed methods for detecting gastric cancer at any stage of its development, by detecting the methylation status of the promoter region consisting of SEQ ID NO:1. In one embodiment, aberrant methylation was detected in 150/198 (76%) of gastric tumors, and 11/20 (55%) of intestinal metaplasia, a precancerous lesion of gastric cancer, but not in 23 normal gastric tissues. Thus it can be seen that methylation of this sequence of SEQ ID NO:1 may be used as a marker for the gastric cancer cells.

The methods may comprise collecting cell samples or taking biopsy samples from a subject, purifying or partly purifying the DNA from the sample, and analysing the portion of the DNA that is greater than about 95%, 96%, 97%, 98%, 99% or more identical to a portion of RNF180 promoter presented as SEQ ID NO:1 to determine its methylation state. Such portions may be 10 nucleotides long or may be more than about 10, 15, 20, 25, 30 or more nucleotides long. In alternative embodiments the methods may further comprise using suitable pairs of sequence specific primers to amplify a portion of the promoter, and may comprise using reagents that react differently with methylated and unmethylated DNA.

The methylation of disclosed sequences may be detected by COBRA or bisulfite sequencing assay using the primers according to FIG. 7 (without using probe).

In embodiments the promoter region of the RNF180 gene considered may be further restricted to the core sequences presented in FIG. 12 as SEQ ID NO:42.

In particular alternative embodiments the method may comprise the steps of determining whether a target sequence of at least 10 consecutive base pairs, within a contiguous sequence at least 99% similar to the region consisting of SEQ ID NO:1 is significantly methylated in the sample; and using this information to thereby detect gastric cancer in the sample. In alternative variants of the embodiment the target sequence may be at least 10, 15, 20, 25, 30, 35, 40, 45, or 50 base pairs long and may contain one or a plurality of CpG base pairs. In particular alternative embodiments the method may further comprise determining whether the sequence is significantly more methylated than in a sample of non-cancerous cells. In particular alternative embodiments the determining may comprise treating the sample with a reagent that differentially modifies methylated and unmethylated DNA, which reagent may be or may comprise a restriction enzyme that preferentially cleaves unmethylated DNA. In embodiments determining may comprise treating the sample with sodium bisulphate and the determination may be performed by combined bisulfite restriction analysis (COBRA) or bisulfite sequencing.

In alternative embodiments of the method the sample may be a blood sample. In further alternative embodiments determining may comprise the steps of: a) treating the DNA from the sample with a restriction enzyme; and b) amplifying the treated DNA using primers selective for a CpG-containing genomic sequence, wherein the genomic sequence is contained within SEQ ID NO:1; and c) comparing the level of the amplified portion of the genomic sequence to determine whether the sequence is significantly more methylated than in a non-cancerous sample to thereby detect the presence of gastric cancer. In embodiments the amplifying uses the polymerase chain reaction.

### Fifth Embodiment

In a fifth embodiment there are disclosed methods for detecting gastric cancer at any stage of its development, by detecting a significantly decreased level of expression of the RNF180 mRNA Transcript variants 1 and 2. The methods may comprise collecting cell samples or taking biopsy samples from a subject, purifying or partly purifying the mRNA from the sample, and analysing the portion of the mRNA that is greater than about 95%, 96%, 97%, 98%, 99% or more identical to a portion of the RNF180 RNA sequence presented as SEQ ID NO:2 to determine its expression level relative to non-cancerous cells. Such portion of SEQ ID NO:2 may be or may be up to or more than about 10, 15, 20, 25, 30, 35, 40, 45 or 50 nucleotides long. In embodiments a significantly increased expression level of the mRNA sequence relative to non-cancerous cells may be taken as indicating the presence of a gastric cancer cells. In alternative embodiments the methods may further comprise making cDNA and using suitable pairs of sequence specific primers to amplify a portion of the cDNA corresponding to the coding sequence presented as SEQ ID NO:2. In embodiments the level of expression of the mRNA transcripts may be assessed by hybridising mRNA to probes suitable to hybridise at high stringency to the coding sequence presented as SEQ ID NO:2.

Specific embodiments of a primer pair suitable for amplifying a segment of the RNF180 RNA to assess expression levels are presented in FIG. 5 as SEQ ID NOS:28 and 29. In alternative variants of the embodiment there is disclosed a method for detecting gastric cancer in a biological sample, said method comprising the steps of: a) detecting the level of a target RNA with at least 99% sequence identity to a region of at least 25 contiguous bases of sequence SEQ ID NO:2 contained in the sample, and b) detecting whether there is a significantly lower amount of the said sequence in the sample relative to a non-cancerous control sample to thereby detect gastric cancer in the biological sample. In particular alternative embodiments. In embodiments the region may be at least 50 base pairs long. In embodiments the sample may be a blood sample. In embodiments the detecting may comprise amplifying the region and the amplifying may use the polymerase chain reaction.

### Sixth Embodiment

In a sixth embodiment there is described a method for suppressing or inhibiting the formation, growth or survival of gastric cancer cells. The method may comprise expressing the RNF180 mRNA or protein or an active portion of either of the foregoing, in a target cell to thereby maintain the non-cancerous state of the cells. The method may comprise introducing into the target cells an expression vector adapted to express the mRNA coding for a protein product of the RNF180 gene.

In embodiments the expression vector may comprise the sequence consisting of Genbank accession Number NM_{—}001113561 (from nucleotide 111 to 1889) representing RNF180 Transcript Variant 1, or the sequence consisting of Genbank accession number: NM_{—}178532 (from nucleotide 111 to 1361) representing RNF180 Transcript Variant 2. The Expression vector may also comprise any suitable sequence suitable to encode a protein that is greater than 95%, 96%, 97%, 98%, or 99% identical to the protein sequence encoded by RNF180 transcripts. FIGS. 11 and 12 show the encoded protein sequences of variant transcripts 1 and 2 respectively (the proteins being referred to as isoforms 1 and 2 respectively). In embodiments an expression vector may contain suitable coding sequences and be adapted to express one or both isoforms, or alternative isoforms, or active portions of any of the foregoing.

In alternative embodiments there are disclosed compositions comprising the expression vectors for use in the method for suppressing gastric cancer and methods for delivering them to a subject.

In particular variants of the embodiment there is disclosed a composition for suppressing gastric cancer, the composition comprising a biologically acceptable expression vector for expressing a portion of the RN180 gene in cells of said gastric cancer. In alternative variants the vector is suitable to direct expression of RNF180 protein in the cells. In alternative variants the method comprises exposing the cells of said subject to the composition. In alternative variants the vector is delivered by viral transduction.

### Seventh Embodiment

In a seventh series of embodiments the levels of expression of RNF180 RNA, or protein, or levels of promoter methylation, may alternatively or in combination be used to predict the likely mortality of a subject or to determine the degree of progress of gastric cancer in the subject. In embodiments patient samples may be analysed to determine levels of any of these variables and the results compared with standard curves to make predictions.

### Eighth Embodiment

In an eighth series of embodiments there are disclosed kits for detecting gastric cancer in a biological sample. The kits may use primers or probes for the RNF180 promoter region, or the RNF180 mRNA, or the RNF180 protein and may include reagents for detecting the levels of expression of the RNF180 mRNA or reagents for detecting the degree of methylation of the RNF180 promoter or the level of expression of RNF180 protein.

In variants of the embodiment there is disclosed a kit for detecting the presence of gastric cancer cells in a biological sample, said kit comprising reagents for detecting a significant level of DNA methylation in a nucleic acid sequence having at least 95% sequence identity over a fragment of SEQ ID NO:1. The fragment may be 10 bases long or may be about up to or more than about 15, 25, 30, 35, 40, 45, 50 or more bases long. The kit may also include instructions to use said reagents to detect the presence of gastric cancer in said sample. In variants of the embodiment the kit may comprise a pair of amplification primers suitable for use to detect RNA transcripts in the sample with at least 95% sequence identity to a sequence of at least 50 contiguous bases of SEQ ID NO:2; and instructions to use said reagents to detect the presence of gastric cancer in said sample. In alternative embodiments the kit may comprise an oligonucleotide probe which binds under high stringency conditions to an isolated nucleic acid having at least 95% sequence identity over at least about 10, 15, 20, 25, 30, 35, 40, 45 or 50 contiguous base pairs to SEQ ID NO:1; and a container for said probe wherein the probe can be used to detect the presence of gastric cancer cells in a biological sample. In alternative embodiments the kit may comprise: an oligonucleotide probe which binds under high stringency conditions to an isolated nucleic acid having at least 95% sequence identity over 10 contiguous base pairs to SEQ ID NO:1 or SEQ ID NO:42; and a container for said probe wherein the probe can be used to detect the methylation of said sequence and thereby the presence of gastric cancer cells in a biological sample. A variety of suitable probe and primer sequences are disclosed in the Examples. In embodiments the level of sequence identity may be more than about 95%, 96%, 97%, 98% or 99% or may be 100%.

### Further Alternative Embodiments

In alternative embodiments there is disclosed a pair of amplification primers suitable to specifically amplify a nucleic acid fragment at least 30 base pairs long and 99% identical to: a) a portion of SEQ ID NO:2 or its complement; or a portion of SEQ ID NO:1 or its complement. In further embodiments the fragment of nucleic acid may be at least about 20, 25, 30, 35, 40, 45, 50 or more bases long, and at least 95%, 96%, 97%, 98%, 99% or 100% identical to a portion of SEQ ID NO:1 or SEQ ID NO:2 or the complements thereof. There are also disclosed expression vectors comprising the sequences and methods of using the sequences to detect or predict or suppress gastric cancer and to give a prognosis for such cancer.

In a further alternative embodiment the methods of other embodiments may be used to predict the development of a gastric cancer, to detect precancerous cells for gastric cancer, or to establish the likely prognosis for gastric cancer.

Examples of suitable primer pairs are presented in the Examples.

### EXAMPLES

The following are examples that illustrate materials, methods, and procedures for practicing the subject matter disclosed. It should be understood that the examples and embodiments described herein are for illustrative purposes only and that various modifications or changes in light thereof will be suggested to persons skilled in the art and are to be included within the spirit and purview of this application.

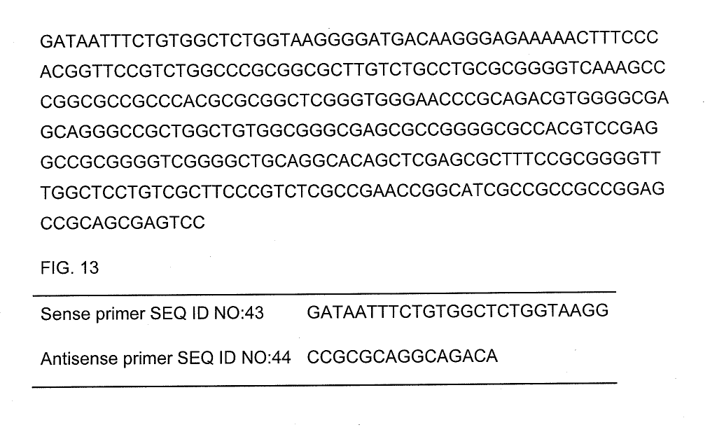

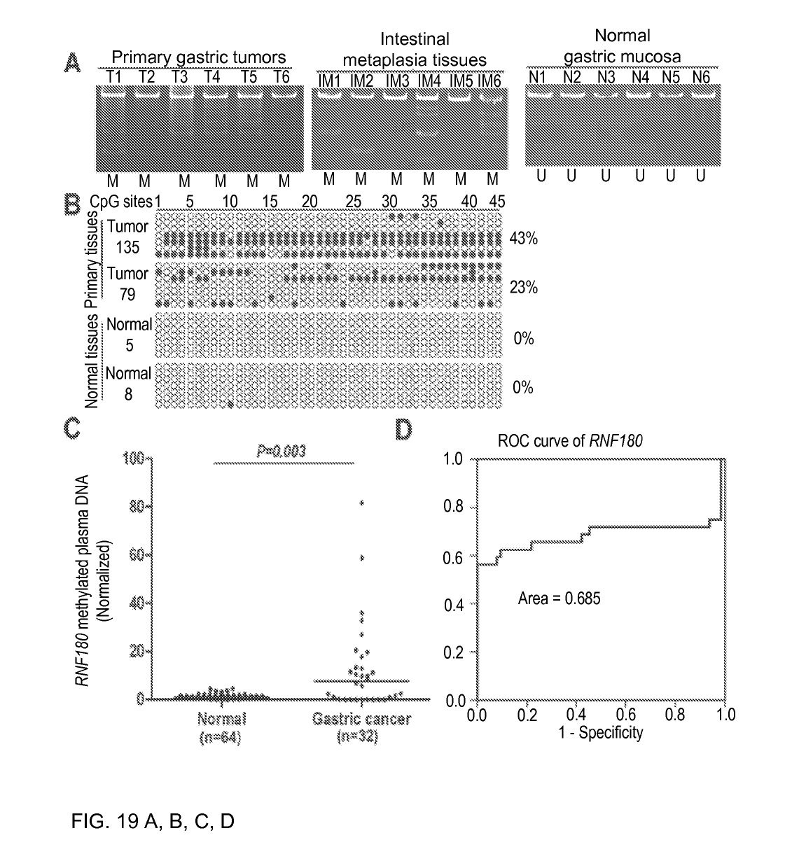

The methylation status of plasma DNA was evaluated using real-time methylation-specific PCR (MS-PCR), while well known and readily understood by those skilled in the art, this technique is also described in Chan et al. Hypermethylated RASSFIA in maternal plasma: A universal fetal DNA marker that improves the reliability of noninvasive prenatal diagnosis. Clinical Chemistry (2006), volume 52, page 2211-2218. Firstly, the plasma DNA (35 μL) was digested with 100 Units of BstUI restriction enzyme (New England BioLabs) at 60° C. for 16 hours. Methylation of RNF180 promoter region, spanning −234 to −144 relative to the TSS, was detected with 2.5 pmol Taqman Probe (Applied Biosystems) in a total volume of 25 μL reaction containing 1× Taqman Universal PCR Master Mix (Applied Biosystems), 200 nmol/L each of RNF180 primers and 7.15 μL DNA digest as a PCR template. The RNF180 methylated plasma DNA levels was calculated using relative quantification. Primers and probe are listed in FIG. 13, SEQ ID NOS:43 and 44 and FIG. 15, SEQ ID NO:46. As revealed by the Receiver operating characteristic (ROC) analysis, the methylated DNA level of plasma samples above the cutoff value of 2.2 will be considered at high susceptibility (91% of specificity) to gastric cancer (FIG. 19C).

### 1. Structure and Analysis of an RNF180 Locus

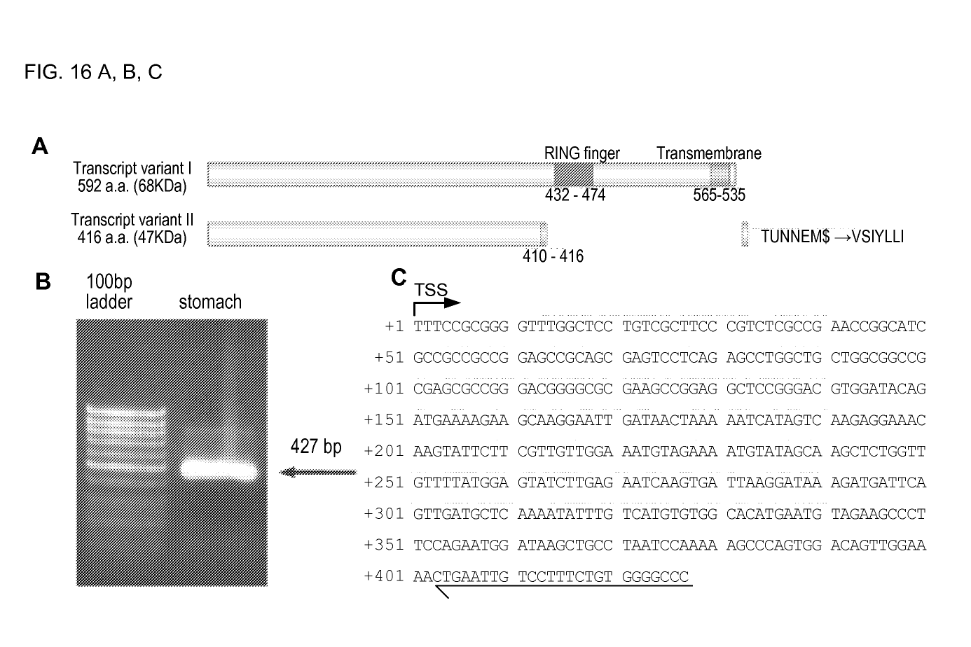

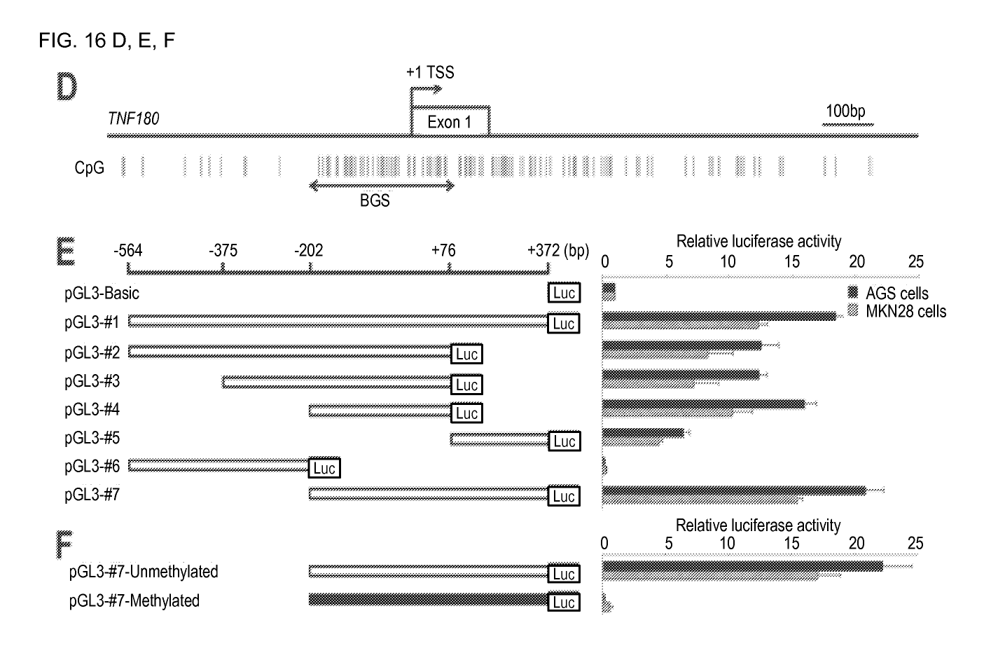

FIG. 16 shows the structure of RNF180 transcript variants, transcription start site (TSS) and functional promoter mapping. (A) The transcript variant 1 (GenBank accession: NP_{—}001107033) contains RING finger and transmembrane domain; the transcript variant 2 (GenBank accession: NP_{—}848627) has a shorter distinct C-terminus and contains no protein domain, lacks multiple 3′ coding exons and differs in the 3′ UTR (TLNNEMS→VSIYLLI; SEQ ID NOS:48 and 49). (B) Gel image of 5′-RACE analysis of RNF180 locus exon 1 starting transcripts. The major PCR band was shown with the product size of 427 bp. (C) Nucleotide sequence of the 5′-RACE products (SEQ ID NO:47). TSS is marked with a bent arrow and assigned as +1. The gene specific reverse primer used for the last PCR is underlined. (D) Diagram shows the location of CpG dinucleotides (vertical bars) of RNF180. The region of COBRA and bisulfite genomic sequencing (BGS) is underlined. (E) Analysis of RNF180 promoter activity by luciferase reporter assay in AGS and MKN28 cells. Different portions of the unmethylated RNF180 promoter (from pGL3-#1 to pGL3-#7) were tested for luciferase activity. Relative luciferase activity is shown for each construct relative to the activity of the pGL3 basic control vector. The above gene diagram and constructs are drawn up to scale. (F) In vitro methylation of the RNF180 promoter region inhibits promoter activity. Luciferase assays in AGS and MKN28 cells with either methylated or unmethylated of the RNF180 construct pGL3#7 was measured. Error bars indicate standard deviation (SD).

### 2. Identification of Transcription Start Site and Core Promoter Region of RNF180 Promoter

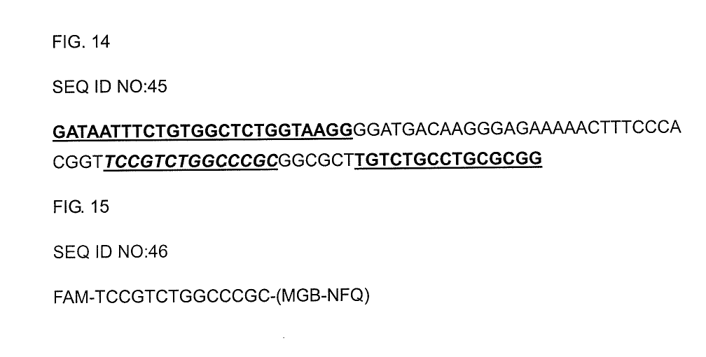

a) 5′ Rapid Amplification of cDNA Ends (5′-RACE)5′-RACE was performed using the GeneRacer Kit with 2 μg of human stomach total RNA according to the manufacturer's instruction (Invitrogen, Carlsbad, Calif., USA). The transcription start site (TSS), defined by the representing 5′-ends, was numbered +1.
b) Construction of RNF180 Luciferase Vectors and Dual-Luciferase Reporter Assay Based on result from 5′-RACE and the region CpG island, seven luciferase constructs (−564 bp to +372 bp) spanning RNF180 promoter and exon 1 were designed. Seven RNF180 inserts tagged with SacI and HindIII were ligated into the pGL3 basic vector. (Promega, Madison, Wis., USA) and were sequence verified. Gastric cancer cell lines (MKN28 and AGS) were seed at a density of 1×10⁵ cells in 24-well tissue culture plates 24 hours before transfection. For each well, we co-transfected 1 μg of RNF180 luciferase vector and 12.5 ng of pRL-CMV internal control vector (Promega) using FUGENE 6 according to the manufacturer's instruction (Roche, Indianapolis, Ind., USA). After 48 hours of transfection, luciferase activity was measured using the Dual-Luciferase Reporter Assay System (Promega). Assays were carried out in three independent triplicates. This assay showed that the promoter region, located −202 bp to +372 bp, exhibited the maximal promoter activity and was defined as the core promoter region now shown as SEQ ID NO:1, in FIG. 1.
c) In vitro DNA Methylation Analysis. Twenty micrograms selected promoter plasmid from the core promoter region were digested with or without 60 U SssI (CpG) methylase overnight (methylated or mock-methylated, respectively) (New England BioLabs, Ipswich, Mass., USA). Methylated and mock-methylated promoter plasmids were purified using illustra GFX PCR DNA and Gel Band Purification Kit (GE Healthcare, Buckinghamshire, England) and their methylation status were verified by the methylation-sensitive restriction enzyme Hpall (New England BioLabs). Luciferase reporter activity was measured in the methylated and mock-methylated transiently transfected MKN28 and AGS cells. The promoter activity of the methylated construct was virtually silenced in both AGS (139-fold reduction) and MKN28 cells (30-fold reduction) when compared to the unmethylated construct. A suitable primer pairs for amplifying a region of the promoter for analysis is presented in FIG. 7 as SEQ ID NOS:35 and 36. FIG. 13 shows an alternative primer pair to amplify a portion of the promoter sequence of FIG. 1. FIG. 14 shows the relationship of the primer sequences in a portion of SEQ ID NO:1.

### 2. Gene Differentially Methylated in Precancerous and Cancerous Gastric Tissues

a) Blood plasma samples were tested from selected patients. A total of 198 patients confirmed gastric cancer, 20 intestinal metaplasia and 23 normal gastric tissues were. Tumor was staged according to the American Joint Committee on Cancer TNM System. All specimens were immediately snap frozen in liquid nitrogen and stored at −80° C. until processing. All subjects gave informed consent and the study protocol was approved by the Clinical Research Ethics Committee of the Chinese University of Hong Kong.
b) Combined Bisulfite Restriction Analysis (COBRA): Genomic DNA was extracted using the DNeasy Tissue Kit (Qiagen™, Valencia, Calif., USA). Sodium bisulfite treated DNA was prepared using the EZ DNA methylation Kit according to the manufacturer's instruction (Zymo™ Research, Hornby, Canada). The methylation level of the RNF180 in gastric biopsies was determined by COBRA. It allows semi-quantitate the methylated and unmethylated DNA by BstUI restriction enzyme digestion on CG dinucleotides (5′-CGCG-3′). After sodium bisulfite conversion, the sequence 5′-CGCG-3′ was retained in the bisulfite-modified methylated DNA while the unmethylated DNA was converted to 5′-UGUG-3 and recognized as 5′-TGTG-3 after PCR amplification. PCR product spans promoter and exon 1 (−207/+94 relative to TSS) which contained 43 CpG dinucleotides and 6 BstUI restriction sites. BstUI cleaved the methylated sequence 5′-CGCG-3′, but not the unmethylated DNA. The PCR digests were resolved on non-denaturing 10% polyacrylamide gels. Promoter methylation of RNF180 was detected in 55% ( 11/20) of gastric precancerous lesion (intestinal metaplasia) and in 76% ( 150/198) of primary gastric tumors, but not in 23 normal gastric tissues.
c) Cloned Bisulfite Genomic Sequencing To further confirm the COBRA results, cloned bisulfite sequencing was performed to identify the methylation status of 43 CG dinucleotide sites. The PCR products from three gastric cell lines, two gastric tumors and two normal gastric tissues, were cloned into the pCR2.1 vector (Invitrogen). Plasmid DNA from the seven PCR clones of each sample was extracted using the QIAprep Spin Miniprep Kit (Qiagen) and sequenced using the T7 promoter primer and M13 primers. Sequence analysis was performed using SeqScape software (Applied Biosystems, Foster City, Calif., USA). The detailed methylation pattern was in accordance with COBRA results.
d) FIG. 19 shows the results promoter methylation of RNF180 analysis in primary tumors and plasma of patients with gastric cancer. (A) Promoter methylation status of RNF180 in primary gastric tumors, intestinal metaplasia tissues and normal gastric mucosa was determined by COBRA. The methylation status was indicated in the representative samples. (8) Cloned bisulfite genomic sequencing analysis of the RNF180 promoter on primary tumors and normal gastric mucosa. (C) Detection of RNF180 methylated plasma DNA in 32 gastric cancer patients and 64 health normal donors. Horizontal bar indicates the median fold change of each sample group. P values were calculated by Mann-Whitney U test. (D) Receiver operating characteristic (ROC) analysis using plasma RNF180 promoter methylation for discriminating gastric cancer.
3. Gene Differentially Methylated in a Biological Sample from Plasma
a) RNF180 Methylated Plasma DNA Detection: Plasma samples were collected from 32 gastric cancer patients and 64 normal donors. Plasma DNA was extracted using QIAamp™ DNA Blood Mini Kit (Qiagen™). Plasma DNA were digested with BstUI enzyme at 60° C. for 16 hours. The methylation of core promoter region from plasma samples was quantitated using real-time methylation-specific PCR with Taqman™ Probe (Applied Biosystems). The RNF180 methylated plasma DNA levels was calculated using relative quantification. The RNF180 methylated DNA level was significantly elevated in plasma samples from patients with gastric cancer compared to those in normal controls (median level 7.6 vs 0.7, P=0.003). The sensitivity was 63% and specificity was 91% at the cutoff value of 2.2 as analyzed by the receiver operating characteristic (ROC) curve (ROC areas: 0.685, 95% Cl=0.54 to 0.84).

### 4. Gene Differentially Expressed in Gastric Cancer

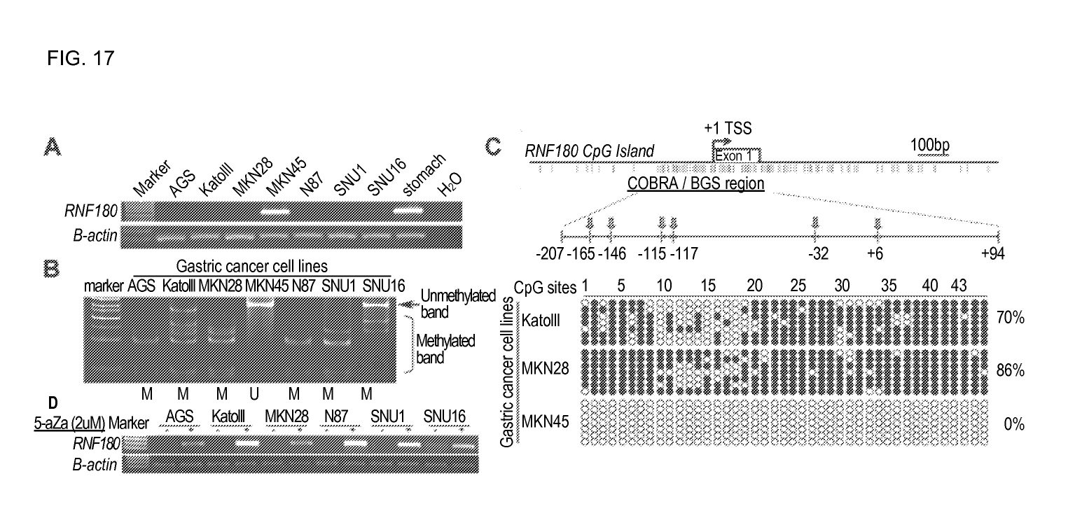

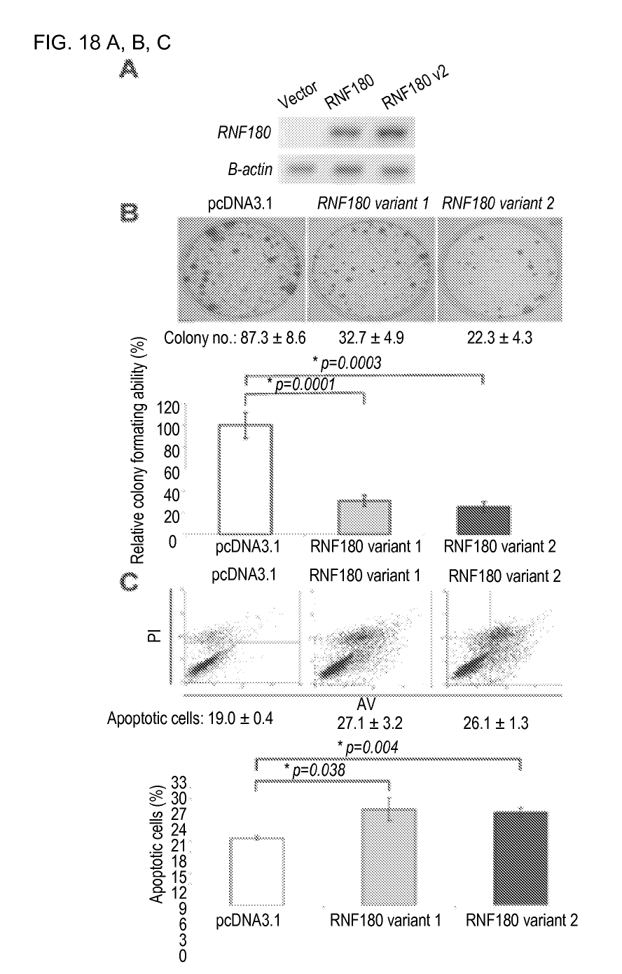

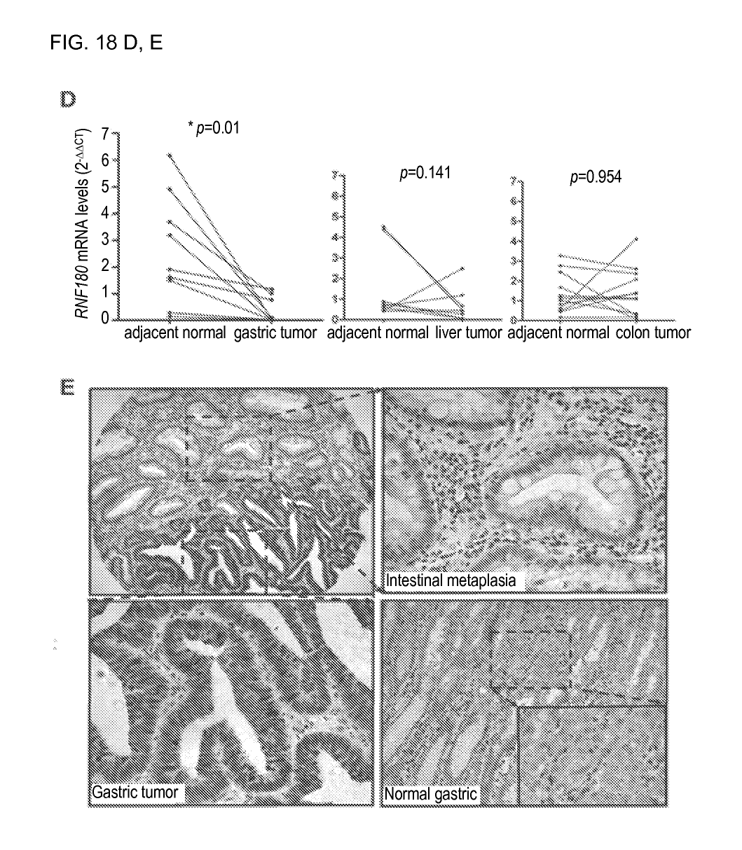

a) FIG. 17 shows mRNA expression and promoter methylation of RNF180 in gastric cancer cell lines. (A) The mRNA expression of RNF180 in seven gastric cancer cell lines and a normal stomach was determined by RT-PCR. (B) COBRA analysis of the RNF180 in gastric cancer cell lines. The undigested fragment (upper band) corresponds to unmethylated DNA, whereas the digested fragments correspond to methylated DNA (lower bands). The methylation status of each sample was indicated at the bottom of the figure. M: methylation; U: unmethylation (C) Cloned bisulfite sequencing analysis of the RNF180 promoter on gastric cancer cell lines. The genomic structure of the RNF180 gene including position of the TSS, CpG dinucleotides, exon 1 and the region of COBRA and bisulfite sequencing (BGS) are labeled. The region of COBRA and BGS spans the core promoter region from −207 to +94. Cleavage of the PCR product with BstUI digestion sites is marked with arrows. A total of seven clones were sequenced per sample. Each row of circles represents sequence analysis of an individual clone. The open circles indicate unmethylated and closed circles indicate methylated CpG site. The overall methylation level in each sample was shown in the right column. (D) RNF180 expression in the GC cell lines following pharmacological reversal of DNA methylation by 5-Aza was examined by RT-PCR.
b) FIG. 18 shows a functional analysis and gene expression of RNF180 in gastric cancer. (A) Expression of RNF180 in RNF180 stably transfected AGS cells was confirmed by RT-PCR. (B) Colony formation assay. Upper panel shows the representative dishes of transfection with pcDNA3.1, RNF180 variant 1 and 2. Quantitative analyses of colony numbers (%) are shown in Lower as values of mean±SD. (C) FACS using Annexin V apoptosis assay. Upper panel shows the FACS histograms of transfection with pcDNA3.1, RNF180 variant 1 and 2. Quantitative analyses of annexin V positive cells (%) are shown in Lower as values of mean±SD. RNF180 mRNA and protein expression in primary paired gastric tumors. (D) Graphical representation of RNF180 expression in 9 paired of human gastric tumors, 8 paired of liver tumors, 12 paired of colon tumors and adjacent non-cancerous tissues were quantified by real-time RT-PCR. Each paired samples were connected with a line. (E) Localization of RNF180 protein assessed by immunohistochemistry on the representative gastric tissue array (Upper-left), intestinal metaplasia (Upper-right), gastric tumor (Lower-left), normal gastric (Lower-right).
c) RNA Isolation and Reverse Transcription PCR Analysis: The RNF180 mRNA levels of 9 pairs of gastric cancers, 12 pairs of colon cancers and 8 pairs of liver tumors were examined. Total RNA was extracted by TRIzol Reagent (Invitrogen). cDNA was synthesized from 2 μg total RNA by Transcriptor Reverse Transcriptase (Roche). mRNA expression was performed using SyberGreen Master Mix (Applied Biosystems) with β-actin as a control. The gene expression data was analyzed using the relative quantification method. RNF180 mRNA expression was significantly downregulated in all (9 out of 9 samples) gastric tumors compared to adjacent non-tumor tissues (P=0.01), but this downregulation was not observed in liver and colon cancers. Suitable primers for selectively amplifying a portion of the RNF180 mRNA sequence for use in the method are shown in FIG. 8.
d) Immunohistochemistry of Gastric Tissues Arrays: Immunohistochemical detection for RNF180 (rabbit anti human, anti-RNF180 polyclonal antibody; Sigma-Aldrich, St Louis, Mo.) was performed on arrays composed of 149 formalin-fixed, paraffin-embedded primary tumor specimens. The slides were examined with a blind test from clinical and other laboratory data. To ensure the accurate assessment of RNF180 protein expression in each tumor, three cores of the same specimens were placed in the tissue arrays. Appropriate positive controls (normal gastric mucosa) were included in each run of immunohistochemistry. RNF180 expression was scored as positive when the staining was strong or moderate; negative when there was weak or no detectable staining compared to positive control. FIG. 9 shows the immunogenic region of predicted RNF180 sequence to which the selected antibody binds.

Our analysis determined 81 (54%) cases were immuno-negative for RNF180 and 68 (46%) cases were immuno-positive for this protein. The immuno-negative group was significantly correlated with TNM stage at advanced stage 1V compared to combined stage I-III (P=0.01).

As shown by the Kaplan-Meier survival curves, immuno-negative patients were significantly correlated with reduced survival (median, 2.70 years) than immuno-positive patients (median, 1.05 years). The proportion of five-year survival rate of immuno-negative patients (16%) was lower than that of RNF180-positive patients (46%) (P=0.0016 by the log-rank test). After stratified tumor staging at stage I-III, this difference was even larger and remained significance. Their five-year survival rate of immuno-negative patients was 13%, as compared with 58% of immuno-positive patients (P=0.0034 by the log-rank test).

To evaluate whether RNF180 as a novel prognostic factor in patients with gastric cancer, all clinical variables available in the dataset (age, gender, H. pylori infection, Lauren type, differentiation or tumor staging) were included in the univariate Cox proportional hazard model. After accounting for potentially confounding factors independent of RNF180 expression, immuno-negative patients was associated with a significantly increased risk of cancer-related death with a hazard ratio of 2.13 (95% confidence interval 1.11 to 4.08; P=0.023) as indicated by a multivariate Cox regression analysis.

### 6. Suppressing Tumor Growth by Restoration of Gene Expression in Gastric Cancer

a) Construction of RNF180 Expression Vectors: The RNF180 expression vectors were generated by PCR-cloning. Briefly, RNA from human stomach (Ambion, Austin, Tex.) was transcribed into cDNA. Sequence corresponding to the open reading frame clone of RNF180 variant 1 and 2 were amplified and verified by DNA sequencing. PCR amplified inserts were subcloned into the pcDNA3.1 TOPO TA expression vector (Invitrogen). Plasmids used for transfection were isolated using EndoFree Plasmid Maxi Kit (Qiagen).
b) Colony Formation Assay: AGS cells were seeded at 1×10⁴ cells on a 24-well plate for 24 hours. Cells were then transfected with 0.4 μg RNF180 transcript and control vector (pcDNA3.1), respectively, using FUGENE 6 (Roche). After 24 hours of post-transfection, cells were subsequently split at 1:10 ratio on six-well plates with RPMI1640 in 10% FBS containing 500 μg/ml neomycin. After 10-12 days of selection, colonies (with >50 cells/colony) were fixed and stained with Giemsa. The experiment was conducted in two independent triplicates. Re-expression of RNF180 transcript significantly suppressed 69% (P=0.0001) colony formation than that of control vector transfected cells.
c) Annexin V Apoptosis Assay: A total of 5×10⁴ AGS cells were seeded on a six-well plate for 24 hr. Cells were then transfected with 2 μg RNF180 transcript and control vector. After 48 hours of transfection, cells were harvested. The proportion of apoptotic cells was evaluated using Annexin V Conjugates for Apoptosis Detection (Invitrogen™) and analyzed using BD FACSCalibur™ System (BD Pharmingen™, San Jose, Calif.). Re-expression of RNF180 transcript induced 43% (P=0.038) of apoptotic cells when compared to controls by annexin-V-FITC/Propidium Iodide using flow cytometry analysis, suggesting that RNF180 had a tumor suppressor property.
d) Statistical Analysis: Student's t-test was analyzed in colony numbers and annexin V positive cells between control and RNF180-transfected cells. Mann-Whitney U test was used for analysis of differences in RNF180 expression in paired tumor/adjacent normal of primary tumors and for analysis of the RNF180 methylated plasma DNA level between gastric patients and healthy normal subjects. The cutoff value, sensitivity and specificity of detecting plasma methylated DNA was analyzed by Receiver Operating Characteristic curve. Data were considered statistically significant when P<0.05. Univariate and multivariate regression from Cox proportional-hazards model was fitted to assess the hazard ratio of RNF180 status and various prognostic variables. Overall survival associated RNF180 status was evaluated by Kaplan-Meier survival curve and log-rank test.
7. Levels of RNF180 Expression are Correlated with Patient Survival.

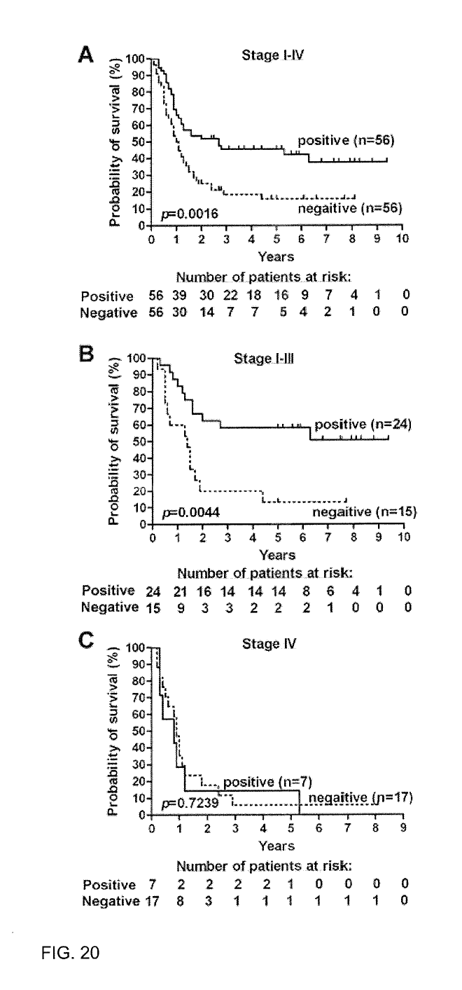

FIG. 20 shows the application of Kaplan-Meier analysis to predict patient survival with gastric cancers, according to RNF180 protein expression. (A) The Kaplan-Meier curve indicates that overall survival of RNF180 immuno-positive and immuno-negative group. The Kaplan-Meier curve of gastric cancer patients is further stratified into (B) the stage I-III and (C) the late stage 1V. The number at risk of each group is indicated. P values were calculated by the log-rank test.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed. Particular embodiments may alternatively comprise or consist of or exclude any one or more of the elements disclosed.

The embodiments of the invention in which an exclusive property or privilege is claimed are defined as follows:

1. A method for detecting gastric cancer in a biological sample, comprising the step of:
detecting methylation of a patient sample sequence of at least 15 consecutive base pairs, within a contiguous sequence at least 95% similar to the region consisting of SEQ ID NO:1;
wherein significant methylation level is indicative of cancer presence in the sample.

2. The method according to claim 1 wherein the target sequence is at least 50 base pairs long and contains a plurality of CpG base pairs.

3. The method according to claim 1 further comprising comparing the methylation level of the patient sample DNA with methylation level of non-cancerous cells.

4. The method according to claim 1, wherein said determining comprises treating the sample with a reagent that differentially modifies methylated and unmethylated DNA.

5. The method according to claim 4, wherein the reagent comprises a restriction enzyme that preferentially cleaves unmethylated DNA.

6. The method according to claim 1 wherein said determining comprises treating the sample with sodium bisulphate.

7. The method according to claim 4, wherein the determination is performed by combined bisulfite restriction analysis (COBRA).

8. The method according to claim 1 wherein the sample is a blood sample.

9. The method according to claim 1 wherein said determining comprises the steps of:
a) amplifying DNA treated with a restriction enzyme using primers selective for a CpG-containing genomic sequence, wherein the genomic sequence is contained within SEQ ID NO:1; and
b) comparing the level of the amplified portion of the genomic sequence in unknown samples to compare the methylation level with non-cancerous sample to thereby detect the presence of gastric cancer.

10. The method according to claim 3, wherein the reagent comprises an enzyme that preferentially cleaves unmethylated DNA.

11. The method according to claim 10, wherein said amplifying uses the polymerase chain reaction.

12. The method according to claim 1 wherein said detecting uses a primer or probe selected from the group consisting of: SEQ ID NOS:3, 4, 5, 6, 7, 8, 9, 10, 11, 12, 13, 14, 15, 16, 17, 18, 19, 20, 21, 22, 23, 24, 25, 26, 27, 35, 36, 43, 44 and 46.

13. A method for detecting gastric cancer in a biological sample, said method comprising the step of:
a) detecting the level of a target RNA with at least 95% sequence similarity to a region of at least 15 contiguous bases of sequence SEQ ID NO:2 contained in the sample,
b) wherein a significantly lower amount of the said sequence in the sample relative to a non-cancerous control sample is indicative of presence of gastric cancer in the biological sample.

14. The method according to claim 13 wherein the region is at least 25 base pairs long.

15. The method according to claim 13 wherein the sample comprises gastric tissue.

16. The method according to claim 13 wherein said detecting comprises amplifying said region.

17. The method according to claim 13 wherein said amplifying uses the polymerase chain reaction.

18. The method according to claim 13 wherein said detecting comprises using a primer or probe selected from the group consisting of: SEQ ID NOS:28, 29, 31, 32, 33, 34, 37 and 38.

19. An isolated nucleic acid sequence at least about 10 base pairs long and 95% identical to a fragment of SEQ ID NO:1, or SEQ ID NO:2.

20. The isolated nucleic acid according to claim 19 wherein said isolated sequence is at least about 20 base pairs long and at least 99% similar to the corresponding fragment of said region.


SEQUENCE LISTING (49 sequences)

SEQ ID NO: 1 - DNA, 606 bp, Homo sapiens
  promoter (1)...(606): region surrounding the core promoter region of ring finger protein 180 (RNF180) gene
gataatttctgtggctctggtaaggggatgacaagggagaaaaactttcccacggttccgtctggcccgcggcgcttgtctgcctgcgcggggtcaaagcccggcgccgcccacgcgcggctcgggtgggaacccgcagacgtggggcgagcagggccgctggctgtggcgggcgagcgccggggcgccacgtccgaggccgcggggtcggggctgcaggcacagctcgagcgctttccgcggggtttggctcctgtcgcttcccgtctcgccgaaccggcatcgccgccgccggagccgcagcgagtcctcagagcctggctgctggcggccgggagcgccgggacggggcgcgaagccggaggctccgggacgtggatacaggtaaaggccggcgggtcggagtcgggcggggcgcggcggcggcgcctctcggagggacctggcctcggccgggccctacccagccgcggtggcccgggcccccacgttggcccaggcggggacgtgccaaggggctgggctagggttgccgctggcctggccgcctctcgcccggcgggcctcaggtgacgcggccgcggcttaactttcgcacctgaggct

SEQ ID NO: 2 - DNA, 191 bp, Artificial Sequence
  synthetic coding sequence common to transcription variants 1 and 2 of RNF180
cctcagaccaggaagagcacctctcccctctggacttcctgcactcagccaatttttcattgggcagcattaatcagaggcttaataagagagaaaggagcaagttgaagaatctaagaaggaaacaacgaaggcgtgaaagatggctacagaagcagggtaaatactcaggagtgggattgctggatcat

SEQ ID NO: 3 - DNA, 14 bp, Artificial Sequence
  synthetic probe for core promoter region of RNF180 gene
tccgtctggcccgc

SEQ ID NO: 4 - DNA, 16 bp, Artificial Sequence
  synthetic alternative probe for core promoter region of RNF180 gene
tggcccgcggcgcttg

SEQ ID NO: 5 - DNA, 16 bp, Artificial Sequence
  synthetic alternative probe for core promoter region of RNF180 gene
tgcctgcgcggggtca

SEQ ID NO: 6 - DNA, 14 bp, Artificial Sequence
  synthetic alternative probe for core promoter region of RNF180 gene
agcccggcgccgcc

SEQ ID NO: 7 - DNA, 15 bp, Artificial Sequence
  synthetic alternative probe for core promoter region of RNF180 gene
acgcgcggctcgggt

SEQ ID NO: 8 - DNA, 16 bp, Artificial Sequence
  synthetic alternative probe for core promoter region of RNF180 gene
cgcagacgtggggcga

SEQ ID NO: 9 - DNA, 16 bp, Artificial Sequence
  synthetic alternative probe for core promoter region of RNF180 gene
ccgctggctgtggcgg

SEQ ID NO: 10 - DNA, 14 bp, Artificial Sequence
  synthetic alternative probe for core promoter region of RNF180 gene
cgagcgccggggcg

SEQ ID NO: 11 - DNA, 16 bp, Artificial Sequence
  synthetic alternative probe for core promoter region of RNF180 gene
acgtccgaggccgcgg

SEQ ID NO: 12 - DNA, 17 bp, Artificial Sequence
  synthetic alternative probe for core promoter region of RNF180 gene
tcgagcgctttccgcgg

SEQ ID NO: 13 - DNA, 18 bp, Artificial Sequence
  synthetic alternative probe for core promoter region of RNF180 gene
tgtcgcttcccgtctcgc

SEQ ID NO: 14 - DNA, 15 bp, Artificial Sequence
  synthetic alternative probe for core promoter region of RNF180 gene
ccggcatcgccgccg

SEQ ID NO: 15 - DNA, 15 bp, Artificial Sequence
  synthetic alternative probe for core promoter region of RNF180 gene
ccggagccgcagcga

SEQ ID NO: 16 - DNA, 16 bp, Artificial Sequence
  synthetic alternative probe for core promoter region of RNF180 gene
tggcggccgggagcgc

SEQ ID NO: 17 - DNA, 15 bp, Artificial Sequence
  synthetic alternative probe for core promoter region of RNF180 gene
cgggacggggcgcga

SEQ ID NO: 18 - DNA, 15 bp, Artificial Sequence
  synthetic alternative probe for core promoter region of RNF180 gene
cggaggctccgggac

SEQ ID NO: 19 - DNA, 16 bp, Artificial Sequence
  synthetic alternative probe for core promoter region of RNF180 gene
aggccggcgggtcgga

SEQ ID NO: 20 - DNA, 15 bp, Artificial Sequence
  synthetic alternative probe for core promoter region of RNF180 gene
agtcgggcggggcgc

SEQ ID NO: 21 - DNA, 15 bp, Artificial Sequence
  synthetic alternative probe for core promoter region of RNF180 gene
cggcggcgcctctcg

SEQ ID NO: 22 - DNA, 14 bp, Artificial Sequence
  synthetic alternative probe for core promoter region of RNF180 gene
ctggcctcggccgg

SEQ ID NO: 23 - DNA, 14 bp, Artificial Sequence
  synthetic alternative probe for core promoter region of RNF180 gene
agccgcggtggccc

SEQ ID NO: 24 - DNA, 16 bp, Artificial Sequence
  synthetic alternative probe for core promoter region of RNF180 gene
aggcggggacgtgcca

SEQ ID NO: 25 - DNA, 15 bp, Artificial Sequence
  synthetic alternative probe for core promoter region of RNF180 gene
ctagggttgccgctg

SEQ ID NO: 26 - DNA, 15 bp, Artificial Sequence
  synthetic alternative probe for core promoter region of RNF180 gene
cgcctctcgcccggc

SEQ ID NO: 27 - DNA, 14 bp, Artificial Sequence
  synthetic alternative probe for core promoter region of RNF180 gene
acgcggccgcggct

SEQ ID NO: 28 - DNA, 20 bp, Artificial Sequence
  synthetic sense primer for amplifying RNF180 gene
cctcagaccaggaagagcac

SEQ ID NO: 29 - DNA, 20 bp, Artificial Sequence
  synthetic antisense primer for amplifying RNF180 gene
atgatccagcaatcccactc

SEQ ID NO: 30 - skipped

SEQ ID NO: 31 - DNA, 20 bp, Artificial Sequence
  synthetic probe for RNF180 gene RNA sequence
acttcctgcactcagccaat

SEQ ID NO: 32 - DNA, 20 bp, Artificial Sequence
  synthetic probe for RNF180 gene RNA sequence
ctctcccctctggacttcct

SEQ ID NO: 33 - DNA, 22 bp, Artificial Sequence
  synthetic probe for RNF180 gene RNA sequence
acttcctgcactcagccaatga

SEQ ID NO: 34 - DNA, 22 bp, Artificial Sequence
  synthetic probe for RNF180 gene RNA sequence
aggagtgggattgctggatcat

SEQ ID NO: 35 - DNA, 25 bp, Artificial Sequence
  synthetic bisulfite sequencing sense primer for amplifying portion of RNF180 gene promoter sequence
atgataagggagaaaaattttttta

SEQ ID NO: 36 - DNA, 24 bp, Artificial Sequence
  synthetic bisulfite sequencing antisense primer for amplifying portion of RNF180 gene promoter sequence
ccaacaaccaaactctaaaaactc

SEQ ID NO: 37 - DNA, 19 bp, Artificial Sequence
  synthetic alternative sense primer for amplifying portion of RNF180 gene mRNA
cgggacgtggatacagatg

SEQ ID NO: 38 - DNA, 22 bp, Artificial Sequence
  synthetic alternative antisense primer for amplifying portion of RNF180 gene mRNA
ccataaaacaaccagagcttgc

SEQ ID NO: 39 - protein, 126 aa, Artificial Sequence
  synthetic RNF180 immunogen region for antibody detection, diagnostic sequence
EKLTLLPTLYEIHSKTTAYSRLNETQPIDLSGLPLQSSKNSYSFQNPSSFDPSMLLQRFSVAPHETQTQRGGEFQCGLEAASVYSDHTNTNNLTFLMDLPSAGRSMPEASDQEEHLSPLDFLHSAN

SEQ ID NO: 40 - protein, 592 aa, Homo sapiens
  ring finger protein 180 (RNF180) isoform 1
MKRSKELITKNHSQEETSILRCWKCRKCIASSGCFMEYLENQVIKDKDDSVDAQNICHVWHMNVEALPEWISCLIQKAQWTVGKLNCPFCGARLGGFNFVSTPKCSCGQLAAVHLSKSRTDYQPTQAGRLMRPSVKYLSHPRVQSGCDKEALLTGGGSENRNHRLLNMARNNNDPGRLTEALCLEVRPTYFEMKNEKLLSKASEPKYQLFVPQLVTGRCATRAFHRKSHSLDLNISEKLTLLPTLYEIHSKTTAYSRLNETQPIDLSGLPLQSSKNSYSFQNPSSFDPSMLLQRFSVAPHETQTQRGGEFQCGLEAASVYSDHTNTNNLTFLMDLPSAGRSMPEASDQEEHLSPLDFLHSANFSLGSINQRLNKRERSKLKNLRRKQRRRERWLQKQGKYSGVGLLDHMTLNNEMSTDEDNEYAEEKDSYICAVCLDVYFNPYMCYPCHHIFCEPCLRTLAKDNPSSTPCPLCRTIISRVFFQTELNNATKTFFTKEYLKIKQSFQKSNSAKWPLPSCRKAFHLFGGFRRHAAPVTRRQFPHGAHRMDYLHFEDDSRGWWFDMDMVIIYIYSVNWVIGFIVFCFLCYFFFPF

SEQ ID NO: 41 - protein, 416 aa, Homo sapiens
  ring finger protein 180 (RNF180) isoform 2
MKRSKELITKNHSQEETSILRCWKCRKCIASSGCFMEYLENQVIKDKDDSVDAQNICHVWHMNVEALPEWISCLIQKAQWTVGKLNCPFCGARLGGFNFVSTPKCSCGQLAAVHLSKSRTDYQPTQAGRLMRPSVKYLSHPRVQSGCDKEALLTGGGSENRNHRLLNMARNNNDPGRLTEALCLEVRPTYFEMKNEKLLSKASEPKYQLFVPQLVTGRCATRAFHRKSHSLDLNISEKLTLLPTLYEIHSKTTAYSRLNETQPIDLSGLPLQSSKNSYSFQNPSSFDPSMLLQRFSVAPHETQTQRGGEFQCGLEAASVYSDHTNTNNLTFLMDLPSAGRSMPEASDQEEHLSPLDFLHSANFSLGSINQRLNKRERSKLKNLRRKQRRRERWLQKQGKYSGVGLLDHMVSIYLLI

SEQ ID NO: 42 - DNA, 310 bp, Homo sapiens
  promoter (1)...(310): synthetic subsequence of core promoter region of ring finger protein 180 (RNF180) gene
gataatttctgtggctctggtaaggggatgacaagggagaaaaactttcccacggttccgtctggcccgcggcgcttgtctgcctgcgcggggtcaaagcccggcgccgcccacgcgcggctcgggtgggaacccgcagacgtggggcgagcagggccgctggctgtggcgggcgagcgccggggcgccacgtccgaggccgcggggtcggggctgcaggcacagctcgagcgctttccgcggggtttggctcctgtcgcttcccgtctcgccgaaccggcatcgccgccgccggagccgcagcgagtcc

SEQ ID NO: 43 - DNA, 25 bp, Artificial Sequence
  synthetic sense primer for methylation detection suitable to amplify RNF180 promoter
gataatttctgtggctctggtaagg

SEQ ID NO: 44 - DNA, 15 bp, Artificial Sequence
  synthetic antisense primer for methylation detection suitable to amplify RNF180 promoter
ccgcgcaggcagaca

SEQ ID NO: 45 - DNA, 91 bp, Homo sapiens
  promoter (1)...(91): synthetic portion of core promoter region of ring finger protein 180 (RNF180) gene
gataatttctgtggctctggtaaggggatgacaagggagaaaaactttcccacggttccgtctggcccgcggcgcttgtctgcctgcgcgg

SEQ ID NO: 46 - DNA, 14 bp, Artificial Sequence
  synthetic Taqman probe sequence for core promoter region of RNF180 gene
  modified_base (1)...(1): t modified by FAM
  modified_base (14)...(14): c modified by (MGB-NFQ)
tccgtctggcccgc

SEQ ID NO: 47 - DNA, 427 bp, Artificial Sequence
  synthetic RNF180 gene 5′-RACE products
tttccgcggggtttggctcctgtcgcttcccgtctcgccgaaccggcatcgccgccgccggagccgcagcgagtcctcagagcctggctgctggcggccgggagcgccgggacggggcgcgaagccggaggctccgggacgtggatacagatgaaaagaagcaaggaattgataactaaaaatcatagtcaagaggaaacaagtattcttcgttgttggaaatgtagaaaatgtatagcaagctctggttgttttatggagtatcttgagaatcaagtgattaaggataaagatgattcagttgatgctcaaaatatttgtcatgtgtggcacatgaatgtagaagcccttccagaatggataagctgcctaatccaaaaagcccagtggacagttggaaaactgaattgtcctttctgtggggccc

SEQ ID NO: 48 - protein, 7 aa, Artificial Sequence
  synthetic RNF180 isoform 1 and isoform 2 3′ UTR difference
TLNNEMS

SEQ ID NO: 49 - protein, 7 aa, Artificial Sequence
  synthetic RNF180 isoform 1 and isoform 2 3′ UTR difference
VSIYLLI